(19) United States
(12) Patent Application Publication
(10) Pub. No.: US 2016/0250118 A1
(43) Pub. Date: Sep. 1, 2016

(54) METHOD AND COMPOSITION TO REMODELLING KERATINIC FIBER

(71) Applicant: ALFA PARF GROUP S.p.A., Bologna (IT)

(72) Inventors: Antonio Consoli, Urgnano (IT); Katiuscia Grevalcuore, Bergamo (IT); Francesco Facheris, Dalmine (IT); Gianluca Borgnini, Carvico (IT)

(21) Appl. No.: 15/051,727

(22) Filed: Feb. 24, 2016

(30) Foreign Application Priority Data
Feb. 26, 2015 (IT) 102015000007041

(51) Int. Cl. A61K 8/36; A45D 7/06; A61K 8/34; A61K 8/31; A61Q 5/04; A61K 8/46

(52) U.S. Cl. CPC A61K 8/36; A61Q 5/04; A61K 8/46; A61K 8/342; A61K 8/31; A45D 7/06; A61K 2800/81

(57) ABSTRACT

Disclosed are compositions in the form of fluid O/W (oil-in-water) or two-phase liquid emulsions for reshaping keratin fibre, comprising:
a) at least one dicarbonyl compound of formula (I), or the hydrates or salts thereof:

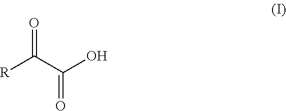

wherein: R represents hydrogen; a carboxyl group; a straight or branched C1-C6 alkyl group optionally substituted by at least one hydroxy or carboxy group or halogen, preferably Br; optionally substituted phenyl; optionally substituted benzyl; an indole or imidazolylmethyl group or the tautomers thereof of formula

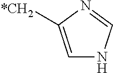

wherein * represents the part connected to the rest of the molecule.
b) at least one lipid selected from a liquid, semisolid or solid hydrocarbon or an ether;
c) at least one ethoxylated or non-ethoxylated fatty alcohol.

### TECHNICAL FIELD OF INVENTION

The present invention relates to cosmetic compositions based on alpha-keto acids and/or derivatives thereof able to reshape keratin fibres, in particular of the hair. The invention also relates to a hair reshaping method using the cosmetic compositions according to the invention.

### PRIOR ART

The cosmetic industry offers a large number of different types of products for treating hair and keeping it healthy. In addition to the common treatments, which comprise products like shampoo, styling products and dyes, hair reshaping products are also becoming popular on the market.

There is growing demand for straight or curly hair, depending on the current fashion, and also to facilitate everyday hair styling. Numerous products able to change the natural shape of the hair are available, some of which can change its shape from straight to curly/wavy, and others vice versa. The latter are known as smoothing or straightening treatments.

One of the major problems associated with the majority of compounds used to straighten wavy, curly, frizzy or kinky hair is that said compounds are highly aggressive towards the hair.

Three general hair reshaping methods have been described over the years. The first two involve significant cleavage of a high percentage of the disulphide bridges deriving from cystine residues. Opening of the disulphide bridges is generally associated with mechanical reshaping of the hair. The third method involves introducing a bridge bond with the aid of aldehydes or carbonyl compounds.

Method 1: Use of Reducing Agents

This method involves a chemical reduction of the cystine structure into two cysteine units, followed by reconfiguration of the hair fibres and re-formation of the cystine structure by oxidation.

The —S—S— bonds of keratin fibre generally maintain the hair in its natural straight or curly configuration. In order to reshape the hair permanently into a different configuration, the disulphide bonds (—S—S—) must be cleaved in significant quantities to form two —SH groups. At this stage, the protein chains of the hair are split from one another, and the hair can be reshaped with the use of specific equipment. Subsequently, by applying hydrogen peroxide, new disulphide bridges are formed. The formation of the new —S—S— bonds has a lasting effect on the shape of the hair in the new configuration. The main ingredient used in this first method of reducing the cystine bonds is ammonium thioglycolate.

Bisulphite and/or sulphite solutions were once, and sometimes still are, used to cleave the disulphide bridges into thiol groups and form a Bunte salt; this method was discovered by Clark and Speakmannel 1932 (see W. Umbach: Kosmetik-Entwicklung, Herstellungund AnwendungkosmetischerMittel, 2nd edition, Georg ThiemeVerlag, Stuttgart, 1995). The reducing composition which contains large amounts of bisulphite and/or sulphite does not have the typical smell of mercaptans.

Fixing is also supported with the aid of heat or oxidising agents, leading to the formation of a new disulphide bridge. In any event, the degree of damage to the hair is high. For this reason, and also for safety reasons, this method is now seldom used.

One of the drawbacks of using reducing agents is that after their application, an oxidising agent such as hydrogen peroxide is used to reconnect the disulphide bridges and deactivate the reducing agent.

Reducing compounds are normally used in an alkaline environment; any excess hydrogen peroxide must therefore be thoroughly removed to prevent hair colour fading problems.

Method 2: Use of Alkaline Agents

Alkaline metal hydroxides have been used to straighten hair for many years. Sodium hydroxide is commonly used as a straightener that provides a permanent reconfiguration. Alkalis (sodium hydroxide, potassium hydroxide and lithium hydroxide) are key ingredients of products called “lyes”.

U.S. Pat. No. 4,304,244 describes guanidine hydroxide as an alternative in products known as “no-lye” straighteners.

Both types of straightener have a pH ranging between 12 and 14.

Hair treatments containing strong bases like hydroxides have a two-step action mechanism. The first step leads to cleavage of the cystine bridges (—S—S—), with the formation of cysteine on the one hand and dehydroalanine on the other, while the second, due to the reaction of cysteine residues with the double bond of the dehydroalanine residue, leads to the formation of a lanthionine bridge (—CH2-S—CH2-), which reconnects the polypeptide chains.

Treatment with hydroxides, in particular alkaline earth hydroxides, leads to the formation of stable, irreversible bonds, with the result that it is impossible to perform a subsequent treatment to change the form of the hair.

Moreover, that composition is very aggressive to the scalp and hair. Even if scalp protectors are used, said compositions can cause strong irritation. A reduction in the elasticity of the hair and, in some extreme cases, even hair breakage and loss, may therefore result. Over exposure to strong alkaline agents can even dissolve the hair. A pre-application diagnosis to establish the health of the hair, and continuous monitoring during the processing time, should therefore be considered. A great deal of research has been conducted to replace hydroxides with less aggressive ingredients. In any event, said compositions were unsatisfactory, in terms of both their straightening effect and their cosmetic effect.

Method 3: Use of Formaldehyde

U.S. Pat. No. 2,390,073 mentions a hair treatment based on the use of formaldehyde or a compound able to release it. According to said document, formaldehyde creates new bridges in the polypeptide structure, which increase the strength of the natural inter-chain bond between two cysteines and generate the straightening effect. The typical concentration used in these types of straightener ranges from 5 to 10% v/v. Subsequently, formaldehyde adducts were marketed in various straightening compositions. The use of formaldehyde is regulated, and its maximum concentration has been set at 0.2% v/v for safety reasons. A considerable amount of formaldehyde evaporates during treatment, because the process requires a hairdryer or hair iron to facilitate the reaction that forms the inter-chain bonds and gives the hair its new shape. In view of the toxicological profile of formaldehyde and its adducts, it has been concluded that its use is unsafe. Many studies have therefore been conducted to replace formaldehyde as a hair straightener. Numerous cosmetics manufacturers have opted for glyoxylic acid.

WO2011104282 discloses a semipermanent hair straightening process that involves applying a solution of alpha-keto acids to the hair for 15 to 120 minutes, then drying and finally straightening the hair with a hair iron at a temperature of approximately 200° C. The preferred alpha-keto acid is glyoxylic acid.

The article “Formaldehyde replacement with glyoxylic acid in semipermanent hair straightening: a new and multidisciplinary investigation” by C. Boga et al. (International Journal of Cosmetic Science, 2014, 36, 459-470) describes the action mechanism of glyoxylic acid and the structure-activity correlations.

However, it has been found that the use of glyoxylic acid presents some drawbacks, especially if the scalp is sensitive and/or irritated. Moreover, glyoxylic acid presents a certain volatility which is increased by the use of the hair iron, and this may cause a further problem during its use. Another problem is that the straightening performance is sometimes insufficient, giving rise to dissatisfaction for those who use this type of straightening. This loss of performance is particularly notable in the case of kinky or very curly hair.

It is also important to note that treatment with a semipermanent straightener gives rise to a colour change and/or colour fading for both natural hair and already dyed hair. This problem leads to dissatisfaction, and the need to perform a further colouring treatment immediately.

The purpose of the invention is to develop a straightening composition that is stable over time and guarantees efficient, persistent hair straightening and/or reduction of volume and frizzy effect. The invention aims at reducing hair damage and to provide a sustainable degree of comfort for the customer and the professional who applies the treatment, minimising colour change and colour fading.

### SUMMARY OF THE INVENTION

It has been found that said purposes are achieved by keratin fibre reshaping compositions comprising a dicarbonyl compound, a lipid and an ethoxylated or non-ethoxylated fatty alcohol. The invention therefore relates to said compositions, their cosmetic use to reshape and straighten the hair, and a hair reshaping method using said compositions.

### DETAILED DESCRIPTION OF THE INVENTION

The compositions according to the invention take the form of fluid 0/W (oil-in-water) or two-phase liquid emulsions, and comprise:
a) at least one dicarbonyl compound of formula (I), or the hydrates or salts thereof:

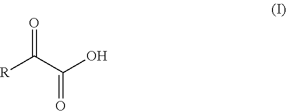

wherein: R represents hydrogen; a carboxyl group; a straight or branched C1-C6 alkyl group optionally substituted by at least one hydroxy or carboxy group or halogen, preferably Br; optionally substituted phenyl; optionally substituted benzyl; an indole or imidazolylmethyl group or the tautomers thereof of formula

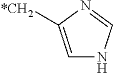

wherein * represents the part connected to the rest of the molecule, in concentrations ranging from 7% to 30% by weight of the total weight of the composition, preferably from 10% to 25% by weight;
b) at least one lipid selected from a liquid, semisolid or solid hydrocarbon or an ether;
c) at least one ethoxylated or non-ethoxylated fatty alcohol.

“Optionally substituted phenyl or benzyl” preferably means a phenyl or benzyl group substituted by a hydroxy or carboxy group.

The compositions according to the invention can optionally be in “ready to use” form, comprising two or more ingredients to be mixed before use.

Said compositions cause a straightening effect that is stable over time and guarantees efficient, persistent hair straightening and/or reduction of volume and frizzy effect. At the same time the invention provides a high degree of comfort for the customer and the professional who applies the treatment, and surprisingly minimises colour change and colour fading.

The preferred compounds of formula (I) comprise glyoxylic acid and derivatives thereof such as esters, amides, thioacetals and hemithioacetals, and amino-acid derivatives known as glyoxyloyl amino acids. Amino acids which can be used to form said derivatives include glycine, aspartic acid, glutamic acid, cysteic acid, cystine and carbocysteine. Glyoxyloyl carbocysteine is particularly preferred.

As an alternative to glyoxylic acid and derivatives thereof, pyruvic acid and/or alpha-ketobutyric acid (2-ketobutyric acid) or derivatives thereof (amides and esters) can be used.

The compounds of formula (I) or derivatives or mixtures thereof are present in the compositions in a sufficient concentration to perform the reshaping effect on the keratin fibre without damaging the fibre.

Examples of hydrocarbons or fatty ethers (ingredients b) comprise isoparaffins, mineral oils, vaselines, paraffins, hydrocarbon waxes (microcrystalline wax, ceresin and ozokerite), cycloparaffin hydrocarbons and terpene hydrocarbons. The preferred compounds are set out below, using the INCI nomenclature: Petrolatum (both solid vaseline and petroleum jelly), paraffin, microcrystalline wax, Ozokerite, Ceresin, C13-14 isoparaffin, C11-12 isoparaffin, C11-13 isoparaffin, C13-16 isoparaffin, isohexadecane, isododecane, isoeicosane, diethylhexylcyclohexane, Squalane and dicaprylyl ether.

Said compounds can be present in the composition in percentages by weight ranging between 20% and 80% of the total weight of the composition, preferably between 25 and 70%.

The fatty alcohol (ingredient c) can be a saturated or unsaturated, straight or branched C8-C30 alcohol, optionally ethoxylated, either alone or in a mixture. One example of a mixture of fatty alcohols is a mixture of cetyl alcohol and stearyl alcohol (cetostearyl alcohol). In the case of ethoxylated fatty alcohols, the ethoxylated moles can range from 1 to 200. The preferred fatty alcohols are set out below, again using the INCI names: caprylic alcohol, ethylhexanol, nonyl alcohol, noneth-8, isononyl alcohol, Decyl Alcohol, Deceth-n (from n=2 to n=10), isodecyl alcohol, isodeceth-n (from n=2 to n=6), undecyl alcohol, undeceth-n (from n=2 to n=40), lauryl alcohol, laureth-n (from n=2 to n=100), isolauryl alcohol, isolaureth-n (from n=3 to n=10), tridecyl alcohol, trideceth-n (from n=2 to n=50), isotridecyl alcohol, myristyl alcohol, myreth-n (from n=2 to n=10), isomireth-n (from n=3 to n=9), pentadecyl alcohol, cetyl alcohol, ceteth-n (from n=1 to n=150), isocetyl alcohol, isoceteth-n (from n=2 to n=30), stearyl alcohol, steareth-n (from n=1 to n=50), isostearyl alcohol, isosteareth-n (from n=2 to n=50), Oleyl Alcohol, Oleth-n (from n=2 to n=110), arachidyl alcohol, arachideth-20, C20-22 Alcohols, C22-24 pareth-33. Decyl Alcohol, Deceth-n (from n=2 to n=10), cetyl alcohol, ceteth-n (from n=1 to n=150), Oleyl Alcohol, Oleth-n (from n=2 to n=110), stearyl alcohol, steareth-n (from n=1 to n=50), isostearyl alcohol, isosteareth-n (from n=2 to n=50) and C20-22 Alcohols are particularly preferred.

Fatty alcohols are present in the composition in percentages by weight ranging from 0.5% to 20% of the total weight of the composition, preferably from about 2% to 15%.

The compositions according to the invention can also contain urea and derivatives thereof in percentages by weight ranging from 0.1% to 10% and/or glycerin or derivatives thereof ranging from 0.1% to 20%.

The compositions according to the invention can also contain other ingredients such as non-ionic, anionic and cationic surfactants.

Examples of non-ionic surfactants which can be used are ethoxylates of fatty alcohols such as PEG-40 castor oil, Coco Glucoside, Decyl Glucoside and Lauryl Glucoside.

Said surfactants can be present in the composition in quantities ranging from 0.5% to 30% by weight of the total weight of the composition, preferably from about 1% to 10%.

The anionic surfactants comprise alkyl sulphates, alkyl ether sulphates, alkyl aryl sulphonates, alpha olefin sulphonates, acyl isethionates, acetyl taurates, acyl sarcosinates, sulphosuccinates, alkoyl polypeptides, acyl glutamates, citric and tartaric acid derivatives, alkyl ether carboxylates in the form of salts of alkaline earth metals, magnesium, ammonium or alkanolamine.

Anionic surfactants can be included in the composition, also in the solid phase.

The preferred anionic surfactants are those identified by the INCI names sodium laurylsulfate, sodium laurethsulfate and ammonium laurethsulfate, The percentages by weight of said surfactants in the compositions according to the invention range from 0.5% to 30% of the total weight of the composition, preferably from about 1% to 10%.

The amphoteric surfactants comprise alkyl betaine, alkylamidopropyl betaine, amphoacetates, amphodiacetates and propionates. The preferred amphoteric surfactants are cocoamidopropyl betaine, commercially available as TegoBetaine CKD (N—N-dimethyl-N-lauroylamidopropyl)-ammoniumacetobetaine), sodium cocoamphoacetate, (DEHYTON G), and disodium dicocoamphoacetate (DEHYTON DC®).

The percentages by weight of said surfactants in the compositions according to the invention range from 0.5% to 30% of the total weight of the composition, preferably from about 1% to 10%.

Cationic surfactants comprise quaternary ammonium salts such as (C10-C24)-alkyldimethylammonium chloride or bromide, preferably di(C12-C18)-alkyldimethylammonium chloride or bromide; (C10-C24)-alkyldimethylethylammonium chloride or bromide; (C10-C24)-alkyltrimethylammonium chloride or bromide, preferably cetyltrimethylammonium chloride or bromide; (C20-C22)-alkyltrimethylammonium chloride or bromide; (C10-C24)-alkyldimethylbenzylammonium chloride or bromide, preferably (C12-C18)-alkyldimethylbenzylammonium chloride; N—(C10-C18)-alkylpyridinium chloride or bromide, preferably N—(C12-C16)-alkylpyridinium chloride or bromide; N—(C10-C18)-alkylisoquinoline or monoalkylsulphate chloride or bromide; N—(C12-C18)-alkylpolyoylaminoformylmethylpyridinium chloride; and the following (indicated by their INCI names): N—(C12-C18)-alkyl-N-methylmorpholinium chloride, bromide or monoalkylsulfate; N—(C12-C18)-alkyl-N-ethylmorpholinium chloride, bromide or monoalkylsulfate; (C16-C18)-alkylpentaoxyethylammonium chloride; diisobutylphenoxyethoxyethyl-dimethylbenzylammonium chloride; salts of N,N-diethylaminoethylstearylamide and -oleylamide and hydrochloric acid, acetic acid, lactic acid, citric acid and phosphoric acid; N-acylaminoethyl-N,N-diethyl-N-methylammonium chloride, bromide or monoalkylsulfate and N-acylaminoethyl-N,N-diethyl-N-benzylammonium chloride, bromide or monoalkylsulphate, wherein the acyl group is preferably stearyl or oleyl. Cetyltrimethylammonium chloride, cetylpyridinium chloride, benzalkonium chloride, benzethonium chloride, dimethyldioctadecylammonium chloride, dioctadecyldimethylammonium bromide and distearoylethyldimonium chloride (DSEDC) are particularly preferred.

The percentages by weight of said surfactants in the compositions according to the invention range from 0.5% to 20% of the total weight of the composition, preferably from about 1% to 10%.

The compositions can also contain non-ionic, cationic, anionic or amphoteric thickening agents or mixtures thereof in percentages by weight ranging from 0.1% to 10% of the total weight of the composition, preferably from about 0.2% to 5%.

The anionic viscosity-controlling agents are, for example, acryloyldimethyltaurate/VP copolymer, ammonium acryloyldimethyltaurate/carboxyethylacrylate crosspolymer and ammonium acryloyldimethyltaurate/Beheneth-25 methacrylate crosspolymer, alginates, polysaccharides, carboxymethylcellulose, alginic acid, sodium alginate, ammonium alginate, calcium alginate, gum arabic, guar gum or xanthan gum, used individually or in combination with other anionic thickening agents, preferably cellulose derivatives and xanthan gum, more preferably xanthan gum.

Examples of cationic thickening agents include hydroxypropyl guar derivatives commonly identified by the INCI name of Hydroxypropyltrimonium Chloride and available on the market under the tradename Catinal CG-100, Catinal CG-200 (Toho), CosmediaGuar C-261N, CosmediaGuar C-261N, CosmediaGuar C-261N (Cognis), DiaGum P 5070 (Freedom Chemical Diamalt), N-HanceCationic Guar (Hercules/Aqualon), Hi-Care 1000, Jaguar C-17, Jaguar C-2000, Jaguar C-13S, Jaguar C-14S, Jaguar Excel (Rhodia), Kiprogum CW and Kiprogum NGK (Nippon Starch). Said class also includes the hydroxypropyl derivatives of hydroxypropyltrimonium guar hydrochloride. Of said derivatives, the polymer identified by the INCI name HydroxypropylGuarHydroxypropyltrimonium Chloride, available on the market under the name of Jaguar C-162 (Rhodia), is preferred.

Other examples are quaternium-18 bentonite, quaternium-18/benzalkonium bentonite, quaternium M-18 hectorite and TEA-hydrochloride.

The non-ionic thickening agents which can be used correspond to the INCI names SPEG-180/Laureth-50 TMMG Copolymer, Butylene Glycol and Polyether-1, and are available from Rockwood under the tradename Pure Thix.

The non-ionic thickening agents can also be polysaccharides, cellulose, methylcellulose, ethylcellulose, hydroxyethylcellulose, methylhydroxyethylcellulose and methylhydroxypropylcellulose, amides and derivatives thereof, xanthan gum and carrageenans.

The polyurethane thickening agents are polymers with a polyurethane base such as polyurethane-30 (LuvigelSTAR®, BASF), or EO/PO block copolymers, such as Pluronics® (BASF) or urethane thickening agents called Dermothix® available from Alzo International Inc. The thickening agents corresponding to INCI names PEG-100 Stearyl Ether Dimer IPDI or PEG-75 Stearyl Ether Dimer IPDI, commercially available under the name of Dermothix, are preferred.

Other additional ingredients of the compositions according to the invention are heat-sensitive polymers such as bis-methoxy PEG-13 PEG-438/PPG-110 SMDI copolymer (ExpertGel® EG 56), bis-methoxy PEG-13 PEG-502/PPG-57/SMDI copolymer (ExpertGel® EG 230), or a mixture thereof, available from Polymer Expert.

The compositions according to the invention can also include solvents, fats, polymers, oligosaccharides and modified oligosaccharides, carbohydrates and derivatives, polyols and derivatives, triglycerides, hydrocarbons, lanolin and derivatives, opacifiers, silicones, hydrolysed proteins, amino acids, complexing agents, UV filters, pigments, preservatives and fragrances.

The water-soluble organic solvents include glycols, glycol ethers and polyols containing 2 to 6 carbon atoms. The glycols are ethylene glycol, propanediols and butanediols. Polyalkyl glycols are, for example, polyethylene glycols, polypropylene glycols, and related products to which ethylene oxide with a molecular weight of up to 1000 D is added.

The organic solvent can constitute up to 30% by weight of the total weight of the composition.

The compositions according to the invention can contain cationic polymers such as Polyquaternium, copolymers of vinylpyrrolidone/N-vinylimidazolium salts (Luviquat®), N-vinylpyrrolidone/dimethylaminoethylmethacrylate copolymers, quaternised with diethyl sulphate, and copolymers of N-vinylcaprolactam/N-vinylpyrrolidone/N-vinylimidazoline salts; cationic cellulose derivatives (Polyquaternium-4 and -10), and acrylamide/diallyldimethylammonium hydrochloride copolymer (Polyquaternium-7). A particular cationic polymer is poly(methacryloyloxyethyltrimethylammonium hydrochloride), known as Polyquaternium-37.

The cationic polymers present in the composition can constitute 0.1% to 5% by weight of the total weight thereof, preferably from about 0.1% to 1%.

The compositions can also include oligosaccharides and modified oligosaccharides; the preferred modified oligosaccharide is Oligoquat®M (INCI name: Stearyl Dihydroxypropyldimonium Oligosaccharides).

Oligosaccharides and modified oligosaccharides can constitute about 0.1% to 10% of the total weight of the composition, preferably from about 0.2% to 5%.

The compositions can also contain silicones (dimethicones, phenylpolysiloxanes, cyclomethicones, etc.) such as the products sold by Dow Corning under the name of Dow Corning® (DC) 556 CosmeticFluid (INCI name: Phenyl Trimethicone), DC 190 (INCI name: PEG/PPG-18/18 Dimethicone), DC 193 (INCI name: PEG-12 Dimethicone), DC 200, DC 1401 (INCI name: Cyclomethicone, Dimethiconol) and DC 1403 (INCI name: Dimethicone, Dimethiconol), and the commercial products DC 244, DC 344 and DC 345 (INCI name: Cyclomethicone), Q2-7224 (Dow Corning; a stabilised trimethylsilylamodimethicone), Dow Corning 929 emulsion (a modified hydroxyamino silicone), SM-2059 (General Electric), SLM-55067 (Wacker), and AbilQuat 3270 and 3272 (Th. Goldschmidt; diquaternary polydimethylsiloxane, INCI name: Quaternium-80).

Silicones are preferably present in quantities ranging from 0.1% to 5% by weight of the total weight of the composition, more preferably from 0.3% to 2.5%.

The composition according to the invention can also include triglycerides; they may be animal products, but are preferably vegetable products such as almond oil, argan oil, avocado oil, calophyllum oil, castor oil, sesame oil, olive oil, jojoba oil, babassu oil, shea butter, linseed oil or sunflower oil.

The triglycerides are preferably present in quantities ranging from 0.1% to 30% by weight of the total weight of the composition, more preferably from 0.1% to 10%.

Lanolin and derivatives thereof can be present in quantities ranging from 0.1% to 30% by weight of the total weight of the composition, more preferably from 0.1% to 10%.

The compositions can also contain opacifiers selected from ammoniostyrene/acrylates copolymers, DEA-styrene/acrylates/DVB copolymer, guanine, mica, styrene/acrylamide copolymer, styrene/acrylates copolymer, and styrene/DVB copolymer; opacifiers having rheological corrector properties such as behenamide, erucamide, Nylon-12, Nylon-66, oleamide, oleyl palmitamide, stearamide, stearamide DEA-distearate, stearamide DIBA-distearate, and stearyl erucamide. Said ingredients can be present in quantities ranging from 0.1% to 3% by weight of the total weight of the composition, preferably from 0.1% to 1%.

The compositions according to the invention can also contain hydrolysed proteins of animal origin, such as hydrolysed proteins obtained from elastin, keratin, silk and milk, possibly in the form of a salt, available on the market under the name of Dehylan® (Cognis), Promois® (SeiwaKasei Co. Ltd.), Collapuron® (Cognis), Nutrilan® (Cognis), Gelita-Sol® (Deutsche Gelatine FabrikenStoess& Co), Lexein® (Inolex) and Kerasol® (Croda).

The proteins or hydrolysed proteins can also be of plant origin, such as proteins or hydrolysed proteins obtained from soy, almonds, peas, potatoes, linseed, corn and wheat, available on the market under the name of Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

The hydrolysed products can also be derivatised, such as Keramimic 2.0® by Croda.

The hydrolysed proteins or derivatives thereof can preferably be present in the composition in quantities ranging from 0.1 to 10% by weight of the total weight of the composition, preferably from 0.1 to 2.5%.

The compositions can also contain amino acids selected from glycine, sarcosine, lysine, serine, glucosamine, glutamic acid, carnitine, acetylcarnitine, alanine, arginine, asparagine, aspartic acid, cysteine, glutamine, histidine, isoleucine, leucine, lysine, methionine, phenylalanine, proline, serine, threonine, tryptophan, tyrosine and valine, preferably arginine, asparagine, glutamine, histidine, lysine, proline and tryptophan.

The amino acids can be present in the composition in quantities ranging from 0.01 to 10% by weight of the total weight of the composition, preferably from 0.01 to 2.5%.

The compositions can also include complexing agents selected from chelating agents, sequestering agents and the salts thereof. Examples of chelating agents include ethylenediaminetetraacetic acid (EDTA), nitrilotriacetic acid, ethyleneglycol-bis(β-amino-ethyl ether)-N,N-tetraacetic acid and ethylenediamine-N,N′-disuccinic acid (EDDS).

The chelating or sequestering agents can be present in quantities ranging from 0.05% to 10% by weight of the total weight of the composition, preferably from 0.05% to 2%.

The compositions can include sunscreens in quantities ranging from 0.01% to 10% of the total weight of the composition.

The compositions can also include a direct non-ionic, cationic or anionic hair dye.

Examples of non-ionic dyes include 2-amino-3-nitrophenol; 2-[(2-hydroxyethyl)amino]-1-methoxy-5-nitrobenzene; 1-(2-hydroxyethoxy)-3-methylamino-4-nitrobenzene; 2,3-(dihydroxyprop oxy)-3-methylamino-4-nitrobenzene; 1-[(2-ureidoethyl)amino]-4-nitrobenzene; 4-[(2-hydroxyethyl)amino]-3-nitro-1-methylbenzene; 1-[(2-hydroxyethyl)amino]-2-nitrobenzene (HC Yellow No. 2); 1-(2-hydroxyethoxy)-2-[(2-hydroxyethyl)amino]-5-nitrobenzene (HC Yellow No. 4); 1-amino-2-[(2-hydroxyethyl)amino]-5-nitrobenzene (HC Yellow No. 5); 4-[(2,3-dihydroxypropyl)amino]-3-nitro-1-trifluoromethylbenzene (HC Yellow No. 6); 1-(4-aminophenylazo)-2-methyl-4-(bis-2-hydroxyethyl)aminobenzene (HC Yellow No. 7); 3-[(2-aminoethyl)amino]-1-methoxy-4-nitrobenzene hydrochloride (HC Yellow No. 9); 1-chloro-2,4-bis-[(2-hydroxyethyl)amino]-5-nitrobenzene (HC Yellow No. 10); 2-[(2-hydroxyethyl)amino]-5-nitrophenol (HC Yellow No. 11); 1-chloro-4-[(2-hydroxyethyl)amino]-3-nitrobenzene (HC Yellow No. 12); 4-[(2-hydroxyethyl)amino]-3-nitro-1-trifluoromethyl-benzene (HC Yellow No. 13); 4-[(2-hydroxyethyl)amino]-3-nitro-benzonitrile (HC Yellow No. 14); 4-[(2-hydroxyethyl)amino]-3-nitro-benzamide (HC Yellow No. 15); 1,4-diamino-2-nitrobenzene; 1,4-bis[(2-hydroxyethyl)amino]-2-nitrobenzene; 2-amino-4,6-dinitro-phenol; 4-amino-3-nitrophenol; 1-amino-5-chloro-4-[(2-hydroxyethyl)amino]-2-nitrobenzene; 4-[(2-hydroxyethyl)amino]-3-nitrophenol (Jarocol NHEAP®); 4-[(2-nitrophenyl)amino]phenol (HC Orange No. 1); 1-[(2-aminoethyl)amino]-4-(2-hydroxyethoxy)-2-nitrobenzene (HC Orange No. 2); 4-(2,3-dihydroxypropoxy)-1-[(2-hydroxyethyl)amino]-2-nitrobenzene (HC Orange No. 3); 2-[(2-hydroxyethyl)amino]-4,6-dinitro-phenol; 4-ethylamino-3-nitrobenzoic acid; 2-[(4-amino-2-nitrophenyl)amino]-benzoic acid; 2-chloro-6-ethylamino-4-nitrophenol; 2-amino-6-chloro-4-nitrophenol; 4-nitro-o-phenylenediamine; 4-[(3-hydroxypropyl)amino]-3-nitrophenol; 2,5-diamino-6-nitropyridine; 1,2,3,4-tetrahydro-6-nitro-quinoxaline; 4-amino-2-nitro-diphenylamine (HC Red No. 1); 4-amino-1-[(2-hydroxyethyl)amino]-2-nitrobenzene (HC Red No. 3); 1-amino-4-[(2-hydroxyethyl)amino-2-nitrobenzene (HC Red No. 7); 1-amino-5-chloro-4-[(2,3-dihydroxypropyl)amino]-2-nitrobenzene (HC Red No. 10); 5-chloro-1,4-[di(2,3-dihydroxypropyl)amino]-2-nitrobenzene (HC Red No. 11); 1-amino-4-[di(2-hydroxyethyl)amino]-2-nitrobenzene hydrochloride (HC Red No. 13); 7-amino-3,4-dihydro-6-nitro-2H-1,4-benzoxazine (HC Red No. 14); 1-amino-3-methyl-4-[(2-hydroxyethyl)amino]-6-nitrobenzene (HC Violet No. 1); 1-(3-hydroxypropylamino)-4-[di(2-hydroxyethyl)amino]-2-nitrobenzene (HC Violet No. 2); 1-(2-hydroxyethyl)amino-2-nitro-4-[di(2-hydroxyethyl)amino]-benzene (HC Blue No. 2); 1-methylamino-4-[methyl-(2,3-dihydroxypropyl)amino]-2-nitrobenzene (HC Blue No. 6); 1-[(2,3-dihydroxypropyl)amino]-4-[ethyl-(2-hydroxyethyl)amino-2-nitrobenzene hydrochloride (HC Blue No. 9); 1-[(2,3-dihydroxypropyl)amino]-4-[methyl-(2-hydroxyethyl)amino]-2-nitrobenzene (HC Blue No. 10); 4-[di(2-hydroxyethyl)amino]-1-[(2-methoxyethyl)amino]-2-nitrobenzene (HC Blue No. 11); 4-[ethyl-(2-hydroxyethyl)amino]-1-[(2-hydroxyethyl)amino]-2-nitrobenzene hydrochloride (HC Blue No. 12); 2-((4-amino-2-nitrophenyl)amino)-5-dimethylaminobenzoic acid (HC Blue No. 13); N,N′-bis(2-hydroxyethyl)-2-nitro-p-phenylenediamine (N,N′-bis(2-hydroxyethyl)-2-nitro-p-phenylenediamine); 1,4-bis[(2,3-dihydroxypropyl)amino]-9,10-anthracenedione (HC Blue No. 14); 1-amino-5-chloro-4-(2,3-dihydroxypropyl)amino-2-nitrobenzene (HC Red No. 10); 1-chloro-2,5-di[(2,3-dihydroxypropyl)amino]-4-nitrobenzene (HC Red No. 11); Isatin.

Non-ionic disperse dyes such as Disperse Black 9, Disperse Red 17, Disperse Violet 1, Disperse Violet 4, Disperse Violet 15, Disperse Violet 27, Disperse Blue 1, Disperse Blue 3, Disperse Blue 7, Disperse Blue 72, and Disperse Blue 377, 2-hydroxyethyl picramic acid, 4-nitrophenyl aminourea, 3-methylamino-4-nitrophenoxyethanol and 2-nitro-5-glycerylmethylaniline, can also be used.

The preferred non-ionic dyes are HC Yellow No. 7, 2-amino-6-chloro-4-nitrophenol, 4-amino-3-nitrophenol, HC Orange No. 1, HC Red No. 1, HC Red No. 3, HC Red No. 13, Disperse Red 17, HC Blue No. 2, 4-[(2-hydroxyethyl)amino]-3-nitrophenol (Jarocol NHEAP®); 4-[(3-hydroxypropyl)amino]-3-nitrophenol (Jarocol Red BN), 4-[ethyl-(2-hydroxyethyl)amino]-1-[(2-hydroxyethyl)amino]-2-nitrobenzene hydrochloride (HC Blue No. 12), 1-(2-hydroxyethoxy)-2-[(2-hydroxyethyl)amino]-5-nitrobenzene (HC Yellow No. 4) and 1-[(2-hydroxyethyl)amino]-2-nitrobenzene (HC Yellow No. 2).

Examples of cationic dyes include Basic Yellow 57, Basic Yellow 87, Basic Brown 16, Basic Brown 17, Basic Orange 31, Basic Orange 69, Basic Red 51, Basic Red 76, Basic Blue 124, Hydroxyanthraquinoneaminopropylmethylmorpholinium methosulfate, HC Blue 15, HC Blue 16 and HC Blue 17, Basic Blue 99.

The preferred cationic dyes are Basic Yellow 57, Basic Yellow 87, Basic Brown 16, Basic Brown 17, Basic Orange 31, Basic Red 51, Basic Red 76, HC Blue 15, HC Blue 16, Basic Blue 75, Basic Blue 99 and Basic Blue 124.

Examples of anionic dyes include Acid Black 1 (CI 20470); Acid Blue 1 (CI 42045); Food Blue 5 (CI 42051); Acid Blue 7 (CI 42080); Acid Blue 9 (CI 42090); Acid Blue 74 (CI 73015); Acid Red 18 (CI 16255); Acid Red 27 (CI 16185); Acid Red 33 (CI 17200); Acid Red 40 (CI 18070), Acid Red 52 (CI 45100), Acid Red 87 (CI 45380); Acid Red 92 (CI 45410); Acid Orange 7 (CI 15510); Acid Violet 43 (CI 60730); Acid Yellow 1 (CI 10316); Acid Yellow 3 (CI 47005); Acid Yellow 23 (CI 19140); Food Yellow 8 (CI 14270), Acid Green 25, D&C Black No. 2, D&C Black No. 3, FD&C Blue No. 1, D&C Blue No. 4, D&C Brown No. 1, FD&C Green No. 3, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 27, D&C Red No. 30, D&C Red No. 31, D&C Red No. 34, D&C Red No. 36, FD&C Red No. 40, Ext.D&C Violet No. 2, FD&C Yellow No. 6, D&C Yellow No. 7, D&C Yellow No. 8, D&C Yellow No. 11.

The preferred anionic dyes are Acid Yellow 1, Acid Yellow 3, Acid Yellow 23, Acid Orange 7, Acid Red 33, Acid Red 40, Acid Red 52, Acid Red 92, Acid Violet 43, Acid Blue 7, Acid Blue 9 and Acid Blue 62.

The dyes can be contained in the composition alone or in mixtures, in quantities ranging from about 0.01 to 4.0% by weight of the total weight of the composition.

The composition can also include natural direct dyes, such as those based on lawsone, juglone, alizarine, purpurine, carminic acid, kermesic acid, purpurogallin, protocatechualdehyde, indigo, isatin, curcumin, spinulosine and apigenidine. Extracts or decoctions containing said natural dyes can also be used.

For further typical dye compounds which can be used in the composition, see the “Dermatology” series, edited by Ch. Culnan and H. Maibach, Verlag Marcel Dekker Inc., New York, Basel, 1986, volume 7, Ch. Zviak; “The Science of Hair Care”, chapter 7, pp. 248-250 (substantive dyes), F. Mearelli “Le piante tintorie nella colorazione dei capelli”, and “European Inventory of Cosmetic Raw Materials”, published by the European Union, obtainable from Bundesverband Deutscher Industrie-und Handelsunternehmen fur Arzneimittel, Reformwaren and Körperpflegemittel e.V., Mannheim.

Pigments such as iron oxides, titanium oxides, zinc oxides, chromium oxides, ultramarine, manganese violet and ferric ferrocyanide can also be used. Other pigments which can be used are those marketed under the name of WATERSPERSE® (S.A COLOR); UNIPURE (SENSIENT); CELLINI® (BASF); DISTINCTIVE® (RESOURCES OF NATURE), COLORONA® (MERCK), WD (DAITO KASEI).

Said pigments can be included in quantities ranging from 0.01 to 10% by weight of the total weight, preferably from 3 to 8%.

The compositions can also include preservatives and fragrances in quantities ranging from 0.01 to 2% by weight of the total weight.

The pH of the composition can range between 0.5 and 3.0, preferably between 0.8 and 2.8, and more preferably between 0.8 and 2.0.

pH correctors can be added in quantities ranging from 0.01 to 10%, preferably from 0.01 to 2%, by weight of the total weight of the composition.

The invention also relates to a hair reshaping method without the use of alkaline or reducing agents, which comprises:

(1) applying the compositions according to the invention;

(2) leaving them to act for 5 to 45 minutes;

(3) reshaping the hair with a heat source.

The hair should preferably be washed with shampoo and dried before coming into contact with the compositions according to the invention.

The heat can be supplied by a hood dryer, hairdryer, straightening iron or other conventional equipment. Heat can be also supplied at stage (2).

The hair can be rinsed and dried before or immediately after reshaping.

In more detail, the method includes the following steps:

(i) washing the hair with shampoo and drying;

(ii) applying the composition according to the invention;

(iii) leaving to act for 5 to 45 minutes, then combing and drying, preferably with a hairdryer;

(iv) reshaping hair with heat and reshaping equipment;

(v) rinsing and drying hair;

(vi) optionally treating hair with a post-treatment.

Alternatively, the method comprises the following steps:

(i) washing hair with shampoo and drying;

(ii) applying the compositions according to the invention;

(iii-a) leaving them to act for 5 to 45 minutes;

(iii-b) a heat source other than a hairdryer (such as a hood) can optionally be used during step (iii-a);

(iv) rinsing hair with tap water;

(v) optionally treating hair with a post-treatment;

(vi) reshaping hair with heat and reshaping equipment.

Step (v) can also be performed immediately after step (iii-b).

The compositions are left to act for approximately 5 to 45 minutes, preferably 15 to 45 minutes, at room temperature or under a heat source. Rinsing and drying prevent the development of smoke, while ensuring the performance of the product. The rinsed and optionally dried hair can then be treated with a straightening iron for 5 to 10 consecutive cycles at a temperature exceeding 100° C., preferably between 150° C. and 250° C., and more preferably between 180° C. and 220° C.

The compositions according to the invention provide a straightening effect that is stable over time and guarantees efficient, persistent hair straightening and/or reduction of volume and frizzy effect. The invention also reduces hair damage and at the same time provides a sustainable degree of comfort for the customer and the professional who applies the treatment. It also minimises colour change and colour fading.

The following examples further illustrate the invention.

The percentages indicated are expressed by weight in relation to the total weight of the composition.

Table 1 illustrates formulas A and D according to the invention, and comparative formulas B, C and E. Formulas A and D are O/W emulsions, while Formula E is an inverse emulsion.

TABLE 1

| | A | B | C | D | E |
|---|---|---|---|---|---|
| Ingredients (INCI) | % | % | % | % | % |
| AQUA (WATER) | q.s. for | q.s. for | q.s. for | q.s. for | q.s. for |
| | 100 | 100 | 100 | 100 | 100 |
| PARAFFINUM | 20 | — | — | 40 | |
| LIQUIDUM | | | | | |
| (MINERAL OIL) | | | | | |
| GLYOXYLIC ACID | 10 | 10 | 18 | 10 | 10 |
| PETROLATUM | 8 | — | — | 2 | |
| CETEARYL ALCOHOL | 6 | — | — | 8 | |
| STEARYL ALCOHOL | 4 | — | — | 7 | |
| CETEARETH-50 | 3 | — | — | 5 | |
| CETEARETH-25 | 2 | — | — | 3 | |
| CYCLO- | | | | | 14.2 |
| PENTASILOXANE | | | | | |
| GLYCERIN | | | | | 10 |
| PEG/PPG-18/18 | | | | | 1 |
| DIMETHICONE | | | | | |

Locks of hair defined as “very curly” were treated with formulas A, B, C and E, using the following method:

(i) wash hair with shampoo and dry;

(ii) apply composition to hair;

(iii) leave to act for 20 minutes at 40° C.;

(iv) rinse hair with tap water;

(vi) treat hair with a post-treatment;

(iv) reshape hair with a hair iron at 200° C.

A score was given by three experts before and after washing, on the basis of how straight or wavy the hair looked. Value 5 corresponds to a perfectly straightened lock of hair with no waves, while value 1 corresponds to the very curly lock before treatment.

TABLE 2
Values obtained from the mean scores given by 3 experts before
and after washing for “very curly” hair.

| | Before | AFTER n WASHES | | | | | | | | | |
|---|---|---|---|---|---|---|---|---|---|---|---|
| Composition | Washing | 1st | 2nd | 3rd | 4th | 5th | 6th | 7th | 8th | 9th | 10th |
| A | 5 | 4.8 | 4.6 | 4.5 | 4 | 3.8 | 3.7 | 3 | 2.9 | 2.8 | 2.8 |
| B | 3.8 | 3.7 | 3.3 | 3.2 | 2.8 | 2.3 | 2 | 2.5 | 2.8 | 2.2 | 2 |
| C | 5 | 5 | 5 | 4.7 | 4.2 | 3.7 | 3.5 | 3.3 | 2.6 | 2.5 | 2.6 |
| E | 4.33 | 4.33 | 4.17 | 3.6 | 3.5 | 3.3 | 3.17 | 2.25 | 2.25 | 2 | 2.1 |

The values shown in Table 2 demonstrate that formula A according to the invention, before washing, obtained the same value as the comparator with the highest concentration of glyoxylic acid (+80%). Moreover, formula A performed much better before washing than formulas B and E, which contained the same concentration of glyoxylic acid. Until the 4th wash Formula C presented the best performance, although it was not very different from that of formula A. Formula B performed worst until the 10th wash. From the 4th to the 10th wash, the composition with the best performance was Formula A.

Table 3 shows the data obtained with the three compositions (A, B and C) reported in Table 1 with locks defined as “kinky”, using the following method:

(i) wash hair with shampoo and dry;

(ii) apply composition to hair;

(iii) leave to act for 20 minutes at 30° C., comb and dry with hairdryer;

(iv) reshape hair with a straightening iron at 220° C.;

(v) rinse hair and apply post-treatment;

(vi) rinse and dry.

TABLE 3
Values obtained from the mean scores given by three experts
before and after washing for “kinky” hair.

| | BEFORE | After n Washes | | | | |
|---|---|---|---|---|---|---|
| COMPOSITION | WASHING | 1st | 2nd | 3rd | 4th | 5th |
| A | 4.5 | 3.67 | 3.67 | 3.33 | 3 | 3 |
| B | 3 | 2.17 | 2.33 | 1.83 | 1.67 | 1.67 |
| C | 3.67 | 2.33 | 2.33 | 2 | 1.83 | 1.67 |

The values shown in Table 3 demonstrate that formula A performs best on kinky hair both before and after washing.

“Kinky” or “Z-pattern” hair is the most difficult to straighten with a semipermanent straightening treatment.

A further test was conducted with composition A by comparison with composition D on kinky hair. The results are shown in Table 4. The following method was used:

(i) wash hair with shampoo and dry;

(ii) apply composition to hair;

(iii) leave to act for 20 minutes at 40° C.;

(iv) treat hair with a post-treatment;

(v) rinse hair with tap water;

(vi) reshape hair with a hair iron at 200° C.

TABLE 4

| COMPOSITION | BEFORE WASHING |
|---|---|
| A | 4.5 |
| D | 5 |

Composition D, which is richest in petrolatum and fatty alcohols, performed better than composition A.

Tests were also conducted on a model, using the compositions shown in Table 5. Composition G is a composition according to the invention, while composition F is a comparator.

TABLE 5

| | F | G |
|---|---|---|
| Ingredients (INCI) | % | % |
| AQUA (WATER) | q.s. for 100 | q.s. for 100 |
| GLYOXYLIC ACID | 10 | 10 |
| DIMETHICONE | 2 | 2 |
| CETEARYL ALCOHOL | 5 | 5 |
| CYCLOPENTASILOXANE | 2 | 2 |
| CETRIMONIUM CHLORIDE | 1 | 1 |
| PROPYLENE GLYCOL | 1 | 1 |
| QUATERNIUM-80 | 0.5 | 0.5 |
| PARFUM (FRAGRANCE) | 0.3 | 0.3 |
| ARGANIA SPINOSA KERNEL OIL | 0.2 | 0.2 |
| POLYQUATERNIUM-7 | 0.5 | 0.5 |
| DISODIUM EDTA | 0.1 | 0.1 |
| PARAFFINUM LIQUIDUM | | 20 |
| (MINERAL OIL) | | |
| PETROLATUM | — | 8 |
| STEARYL ALCOHOL | — | 4 |
| CETEARETH-50 | — | 3 |
| CETEARETH-25 | — | 2 |

One of the models (M1) had “very curly” hair, while the second (M2) had “kinky” hair.

The following method was used:

(i) wash hair with shampoo and dry;

(ii) apply composition to hair;

(iii) leave to act for 20 minutes at 30° C., comb and dry with hairdryer;

(iv) reshape hair with a straightening iron at 200° C.;

(v) rinse hair and apply post-treatment;

(vi) rinse and dry.

The results are shown in Table 6.

| | |
|---|---|
| Before washing | After 5 washes |

| | F | FG | F | G |
|---|---|---|---|---|
| M1 | 4.2 | 5 | 3 | 3.7 |
| M2 | 3.6 | 4.1 | 2.5 | 3 |

It was also surprisingly observed that when composition G was used, hair colour fading was lower.

For this purpose, tests were conducted on a dyed lock of hair to establish the extent of colour fading after straightening treatment with compositions A and B. The initial colours considered were shade 3.0 (dark chestnut) and shade 5.66 (light extra red brown). The results obtained are shown in Tables 7 and 8. The method used was as follows:

(i) wash hair with shampoo and dry;

(ii) apply composition to hair;

(iii) leave to act for 20 minutes at 40° C.;

(iv) rinse hair with tap water;

(vi) treat hair with a post-treatment and rinse;

(iv) reshape hair with a hair iron at 200° C.

TABLE 7
Effect of compositions A and B on hair dyed with shade 5.66.

| | L* | a* | b* | dL* | da* | db* | dE*ab |
|---|---|---|---|---|---|---|---|
| | (D65) | (D65) | (D65) | (D65) | (D65) | (D65) | (D65) |
| Before treatment | 20 | 13.6 | 4.2 | — | — | — | — |
| After treatment with | 27.97 | 19.41 | 9.12 | 7.97 | 5.81 | 4.92 | 11.02 |
| composition A | | | | | | | |
| After treatment with | 36.53 | 17.42 | 11.92 | 16.53 | 3.82 | 7.72 | 18.64 |
| composition B | | | | | | | |

TABLE 8
Effect of compositions A and B on hair dyed with shade 3.0.

| | L* | a* | b* | dL* | da* | db* | dE*ab |
|---|---|---|---|---|---|---|---|
| | (D65) | (D65) | (D65) | (D65) | (D65) | (D65) | (D65) |
| Before treatment | 17.43 | 0.72 | 0.11 | — | — | — | — |
| After treatment with | 22.91 | 5.41 | 6.64 | 5.48 | 4.69 | 6.53 | 9.73 |
| composition A | | | | | | | |
| After treatment with | 24.71 | 6.04 | 8.98 | 7.27 | 5.32 | 8.87 | 12.64 |
| composition B | | | | | | | |

The dyeing results are expressed in colour space values L*a)*b* and were measured with a Minolta CM-2500d colorimeter.

In colour space L*a*b*, L* indicates lightness (low values indicate darker shades, and thus better dye absorption by the keratin fibre), while a* and b* are the colour coordinates. a* and b* indicate the colour directions: +a* is the direction of red, −a* is the direction of green, +b* is the direction of yellow and −b* is the direction of blue.

Differences in colour can be expressed by the values ΔE, which are defined by the following equation: ΔE=[(ΔL*)²+(Δa*)²+(Δb*)²]^{1/2}

wherein

ΔL represents the difference in lightness (low values indicate darker shades, but also better dye absorption by the keratin fibre);

Δa represents the red-green difference;

Δb represents the yellow-blue difference.

As will be seen from the dEab data, the value is lower in both cases for the composition according to the invention. This means that colour fading was lower.

Table 9 shows examples of compositions according to the invention.

TABLE 9

| Ingredients | % | % | % | % | % | % | % | % | % | % | % | % | % | % | % |
|---|---|---|---|---|---|---|---|---|---|---|---|---|---|---|---|
| AQUA (WATER) | q.s. | q.s. | q.s. | q.s. | q.s. | q.s. | q.s. | q.s. | q.s. | q.s. | q.s. | q.s. | q.s. | q.s. | q.s. |
| | for | for | for | for | for | for | for | for | for | for | for | for | for | for | for |
| | 100 | 100 | 100 | 100 | 100 | 100 | 100 | 100 | 100 | 100 | 100 | 100 | 100 | 100 | 100 |
| PARAFFINUM LIQUIDUM | 20 | 20 | 20 | 20 | 20 | 20 | 20 | 20 | 20 | 30 | 40 | 20 | 20 | 20 | 20 |
| (MINERAL OIL) | | | | | | | | | | | | | | | |
| PYRUVIC ACID | 15 | | | 15 | | | 15 | | | | | 10 | 5 | | |
| GLYOXYLIC ACID | | 10 | | | 10 | | | 10 | | | | 5 | 5 | | 10 |
| 2-KETOBUTYRIC ACID | | | 15 | | | 15 | | | 15 | | | | 2.5 | | |
| GLYOXYOYL | | | | | | | | | | | | | 2.5 | 10 | |
| CARBOCYSTEINE and | | | | | | | | | | | | | | | |
| GLYOXYLOYL KERATIN | | | | | | | | | | | | | | | |
| AMINO ACIDS | | | | | | | | | | | | | | | |
| PETROLATUM | 8 | 8 | 8 | 8 | 8 | 8 | 8 | 8 | 8 | 10 | 5 | 8 | 8 | 8 | |
| CETEARYL ALCOHOL | 6 | 6 | 6 | | | | 3 | 3 | 3 | 6 | 6 | 6 | 6 | 6 | 6 |
| ISOHEXADECANE | | | | | | | | | | | | | | | 28 |
| STEARYL ALCOHOL | 4 | 4 | 4 | | | | 4 | 4 | 4 | 4 | 4 | 4 | 3 | 4 | 4 |
| CETEARETH-50 | 3 | 3 | 3 | | | | 3 | 3 | 2 | 3 | 3 | 2 | 2 | 3 | 3 |
| CETEARETH-25 | 2 | 2 | 2 | | | | 2 | 2 | 2 | 2 | 2 | 2 | 2 | 2 | 2 |
| OLETH-20 | | | | 3 | 3 | 3 | | | 2 | 2 | 3 | | | | |
| OLETH-10 | | | | 3 | 3 | 3 | | | | | | | | | |
| OLEYL ALCOHOL | | | | 8 | 8 | 8 | 3 | | | | | | | | |
| DECETH-5 | | | | 2 | 2 | 2 | | | 1 | | | 1 | | | |
| C20-22 ALCOHOLS | | | | 2 | 2 | 2 | | 3 | | | | | 1 | | |

1. Compositions in the form of fluid 0/W (oil-in-water) or two-phase liquid emulsions comprising:
a) at least one dicarbonyl compound of formula (I), its hydrates or salts: 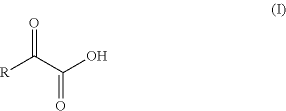
wherein: R is hydrogen; a carboxyl group; a straight or branched C1-C6 alkyl group optionally substituted by at least one hydroxy or carboxyl group or by halogen, preferably Br; optionally substituted phenyl; optionally substituted benzyl; an indole or imidazolylmethyl group or its tautomers of formula 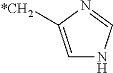
wherein * represents the part bound to the rest of the molecule, in concentrations by weight ranging from 7% to 30% of the total weight of the composition;
b) at least one lipid selected from a liquid, semisolid or solid hydrocarbon, or an ether, in percentages by weight ranging from 20% to 80% of the total weight of the composition;
c) at least one ethoxylated or non-ethoxylated fatty alcohol.

2. Compositions according to claim 1 wherein the dicarbonyl compound of formula (I) is glyoxylic acid, esters thereof, amides, thioacetals, hemithioacetals or derivatives with amino acids.

3. Compositions according to claim 2 wherein the dicarbonyl compound of formula (I) is Glyoxyloyl Carbocysteine.

4. Compositions according to claim 1 wherein the dicarbonyl compound is pyruvic acid or alpha-ketobutyric acid (2-ketobutyric acid) or derivatives thereof.

5. Compositions according to one or more of claims 1 to 4 wherein the hydrocarbons or fatty ethers are selected from isoparaffin, mineral oils, vaseline, paraffins, hydrocarbon waxes, cycloparaffin hydrocarbons and terpene hydrocarbons.

6. Compositions according to one or more of claims 1 to 5 wherein the fatty alcohol is a saturated or unsaturated, straight or branched, optionally ethoxylated C8-C30 alcohol, alone or in mixture.

7. Compositions according to claim 6 wherein the fatty alcohol is selected from Caprylic alcohol, ethylhexanol, nonyl alcohol, noneth-8, isononyl alcohol, Decyl Alcohol, Deceth-n (from n=2 to n=10), isodecyl alcohol, isodeceth-n (from n=2 to n=6), undecyl alcohol, undeceth-n (from n=2 to n=40), lauryl alcohol, laureth-n (from n=2 to n=100), isolauryl alcohol, isolaureth-n (from n=3 to n=10), tridecyl alcohol, trideceth-n (from n=2 to n=50), isotridecyl alcohol, myristyl alcohol, myreth-n (from n=2 to n=10), isomireth-n (from n=3 to n=9), pentadecyl alcohol, cetyl alcohol, ceteth-n (from n=1 to n=150), isocetyl alcohol, isoceteth-n (from n=2 to n=30), stearyl alcohol, steareth-n (from n=1 to n=50), isostearyl alcohol, isosteareth-n (from n=2 to n=50), Oleyl Alcohol, Oleth-n (from n=2 to n=110), arachidyl alcohol, arachideth-20, C20-22 Alcohols, C22-24 pareth-33.

8. Compositions according to claim 6 or 7 wherein the fatty alcohols are present in percentages by weight ranging from 0.5% to 20% by weight of the total composition.

9. Compositions according to one or more of claims 1 to 8 containing one or more ingredients selected from urea, glycerin, non-ionic, anionic or cationic surfactants, non-ionic, cationic, anionic or amphoteric thickening agents, solvents, fats, polymers, oligosaccharides and modified oligosaccharides, carbohydrates and derivatives, polyols and derivatives, triglycerides, hydrocarbons, lanolin and derivatives, opacifiers, silicones, protein hydrolysates, amino acids, complexing agents, UV filters, pigments, preservatives and fragrances.

10. Compositions according to one or more of claims 1 to 9 further containing non-ionic, cationic or anionic direct hair dyes.

11. A hair reshaping method without alkaline or reducing agents, which comprises:
(1) applying the compositions of claims 1-10;
(2) leaving in situ for 5 to 45 minutes;
(3) reshaping hair using a heat source.
